(19) United States
(12) Patent Application Publication
(10) Pub. No.: US 2015/0190484 A1
(43) Pub. Date: Jul. 9, 2015

(54) METHODS FOR REDUCING CELLULAR SENESCENCE MEDIATED BY DNA DAMAGING AGENTS

(71) Applicants: Trustees of Dartmouth College, Hanover, NH (US); The Government of the United States, as represented by the Secretary, Department of Health, Bethesda, MD (US)

(72) Inventors: Mary Jo Mulligan-Kehoe, Enfield, NH (US); Deborah Eva Citrin, Bethesda, MD (US); Eunjoo S. Chung, Rockville, MD (US); James Mitchell, Damascus, MD (US)

(73) Assignees: Trustees of Dartmouth College; The Government of the United States

(21) Appl. No.: 14/661,686

(22) Filed: Mar. 18, 2015

Related U.S. Application Data
(63) Continuation-in-part of application No. PCT/US2014/048604, filed on Jul. 29, 2014.
(60) Provisional application No. 61/867,801, filed on Aug. 20, 2013.

(51) Int. Cl. A61K 38/57

(52) U.S. Cl. CPC A61K 38/57

(57) ABSTRACT

A method for reducing cellular senescence in a subject, in particular a subject exposed to or at risk of being exposed to a DNA damaging agents, e.g., radiation, by inhibiting PAI-1 activity in the subject with a truncated PAI-1 agent, rPAI-1₂₃.

### INTRODUCTION

This application is a continuation-in-part application of PCT/US2014/048604, filed Jul. 29, 2014, which claims the benefit of priority from U.S. Provisional Patent Application Ser. No. 61/867,801, filed Aug. 20, 2013, the contents of which are incorporated herein by reference in their entireties.

### BACKGROUND

TGF-β regulates transcription of numerous genes, including plasminogen activator inhibitor-1 (PAI-1; Eitzman, et al. (1996) J. Clin. Invest. 97:232-237; Loskutoff, et al. (2000) J. Clin. Invest. 106:1441-1443). PAI-1 is the primary inhibitor of the fibrinolytic pathway, thereby contributing to extracellular matrix (ECM) accumulation if its levels are not tightly regulated (Ghosh & Vaughan (2012) J. Cell. Physiol. 227:493-507). Mice that overexpress PAI-1 are sensitive to bleomycin-induced fibrosis, while those that lack the PAI-1 gene are resistant to fibrosis (Eitzman, et al. (1996) J. Clin. Invest. 97:232-237). When radiation and TGF-β treatment are combined, they have been shown to act synergistically to upregulate PAI-1 expression (Hageman, et al. (2005) Clin. Cancer Res. 11:5956-5964), but radiation alone can stimulate PAI-1 expression. T

Studies have been performed, which indicate that PAI-1 has an important role in fibrosis that accompanies radiation therapy in cancer patients, including the cooperative roles of PAI-1 and TGF-β in inducing fibrosis in tissues (Hageman, et al. (2005) Clin. Cancer Res. 11:5956-5965). In addition, it has been shown that PAI-1 is sufficient for the induction of replicative senescence downstream of p53 and that suppression of PAI-1 by RNAi leads to escape from replicative senescence (Kortlever et al. (2006) Nat. Cell Biol. 8:877-884).

Specific PAI-1 inhibitors have been identified, e.g., PAI-749 (Gardell, et al. (2007) Mol. Pharmacol. 72:897-906). Experiments have also been performed in PAI-1 knockout mice (Milliat, et al. (2008) Am. J. Pathol. 172:691-701; Abderrahmani, et al. (2009) Int. J. Radiation Oncology 74:942-948; Abderrahmani, et al. (2012) PLoS ONE 7:e35740). These studies have shown that PAI-1 activity is essential for production of radiation-induced tissue injury in intestinal tissue (Milliat, et al. (2008) Am. J. Pathol. 172:691-701), and that the level of PAI-1 activity is correlated with the severity of radiation-induced intestinal injury in PAI-1 knockout mice (Abderrahmani, et al. (2009) Int. J. Radiation Oncology 74:942-948; Abderrahmani, et al. (2012) PLoS ONE 7:e35740). In addition, the use of a PAI-1 inhibitor, PAI-039, was shown to confer temporary protection against early lethality. In particular, while PAI-039 treatment limited the radiation-induced increase of connective tissue growth factor and PAI-1 at 2 weeks after irradiation, the inhibitor had no effect at 6 weeks.

U.S. Pat. No. 7,951,806, U.S. Pat. No. 5,415,479, US 2009/0124620, US 2011/0112140 and US 2012/0022080 disclose the use of PAI-1 inhibitors to treat diseases, including radiation injury in tissues. The agents disclosed are chemically-synthesized agents and are not variants of PAI-1 itself.

US 2006/0084056 discloses that levels of PAI-1 in a cancer patient can be used to identify patients with poor outcomes. The application also discusses that levels of PAI-1 are related to the level of fibrosis in tissue.

US 2009/0227515 discloses peptides with activity to inhibit PAI-1 and their use to treat fibrotic damage in tissues, including radiation injury.

U.S. Pat. No. 7,241,446, U.S. Pat. No. 7,306,803, and U.S. Pat. No. 7,510,714 are directed to methods for inhibiting angiogenesis. A variant of PAI-1 is taught in these patents, rPAI-1₂₃ (SEQ ID NO:1)_{f} which is a truncated form of PAI-1.

The truncated PAI-1 isoform, rPAI-1₂₃, is a potent angiogenesis inhibitor in vitro, ex vivo (Drinane, et al. (2006) J. Biol. Chem. 281:33336-33344; Mulligan-Kehoe, et al. (2002) J. Biol. Chem. 277:49077-49089; Mulligan-Kehoe, et al. (2001) J. Biol. Chem. 276:8588-8596), and in a mouse model of atherosclerosis (Drinane, et al. (2009) Circ. Res. 104:337-345; Mollmark, et al. (2011) Circ. Res. 108:1419-1428; Mollmark, et al. (2012) Arterioscl. Thromb. Vasc. Biol. 32:2644-2651). Data showed that rPAI-1₂₃ blocks native PAI-1 function in vascular tissue through a novel pathway that increases plasmin activity (Mollmark, et al. (2011) Circ. Res. 108:1419-1428). The plasmin activity degrades key components of the ECM/basement membrane (BM) to result in loss of binding sites for FGF-2 (Mollmark, et al. (2012) Arterioscl. Thromb. Vasc. Biol. 32:2644-2651), a key factor in wound healing, angiogenesis and a potential fibrosis stimulator (Gridley, et al. (2004) Int. J. Radiat. Oncol. Biol. Phys. 60:759-766; Masola, et al. (2012) J. Biol. Chem. 287:1478-1488; Traub, et al. (2012) Int. J. Colorect. Dis. 27:879-884).

### SUMMARY OF THE INVENTION

The present invention is a method for reducing cellular senescence mediated by a DNA damaging agent by administering to a subject in need thereof an effective amount of rPAI-1₂₃. In one embodiment, the subject has been exposed to, is at risk of being exposed to, or will be exposed to a DNA damaging agent. In another embodiment, the DNA damaging agent comprises radiation, a genotoxic agent, a mutagenic agent, or an aging or aging-related disorder. In a further embodiment, the rPAI-1₂₃ has the sequence as set forth in SEQ ID NO:1 or SEQ ID NO:2.

### DETAILED DESCRIPTION OF THE INVENTION

Cellular senescence denotes a stable and long-term loss of proliferative capacity, despite continued viability and metabolic activity. Initially defined by the phenotype of human fibroblasts undergoing replicative exhaustion in culture, senescence can be triggered in many cell types in response to diverse forms of cellular damage or stress. Although once considered a tissue culture phenomenon, studies have demonstrated that cellular senescence imposes a potent barrier to tumorigenesis and has been observed in certain aged tissues or damaged tissues.

It has now been found that rPAI-1₂₃ can protect against cellular senescence, which results from exposure to a DNA-damaging agent. Therefore, the present invention provides a method for reducing cellular senescence mediated by a DNA damaging agent by administering to a subject in need thereof an effective amount of rPAI-1₂₃. The method described herein is useful, e.g., for treating subjects who have been exposed, are suspected to have been exposed, or will be exposed, to radiation, e.g., from a nuclear reactor or a nuclear weapon, from a radiation therapy (e.g., external beam radiation therapy (e.g., x-rays and/or gamma rays) or radioactive pharmaceutical compound), and/or from radioactive materials. Furthermore, DNA damage associated with aging and aging-related disorders or genotoxic or mutagenic agents can also be treated in accordance with this invention.

The term and “subject” and “patient” are used interchangeably throughout the specification to describe an animal, human or non-human, to whom treatment according to the method of the invention is provided. Veterinary applications are contemplated by the present invention. The term includes but is not limited to mammals, e.g., humans, other primates, pigs, rodents such as mice and rats, rabbits, guinea pigs, hamsters, cows, horses, cats, dogs, sheep and goats. The term “treat,” “treatment” or “treating” is used herein to describe delaying the onset of, inhibiting, or alleviating the effects of a condition, e.g., DNA damage, in a patient.

The term “DNA damage” is an art-recognized term and is used herein to refer to chemical changes to DNA, e.g., damaged (oxidized, alkylated, hydrolyzed, adducted, or cross-linked) bases, single-stranded DNA breaks, and double-stranded DNA breaks.

Causative agents of DNA damage (i.e., DNA damaging agents) include, for example, ultraviolet light; ionizing radiation (X-rays, gamma rays, alpha particles); aging and aging-related disorders (e.g., Hutchinson-Gilford Progeria Syndrome, Werner's syndrome, Cockayne's syndrome, or xeroderma pigmentosum); and genotoxic or mutagenic agents, e.g., reactive oxygen species, base analogs, deaminating agents (e.g., nitrous acid), intercalating agents (e.g., ethidium bromide), alkylating agents (e.g., ethylnitrosourea), alkaloids (e.g., Vinca alkaloids), bromines, sodium azide, psoralen, and benzene. Exemplary genotoxic agents used in cancer therapy include busulfan, bendamustine, carboplatin, carmustine, chlorambucil, cisplatin, cyclophosphamide, dacarbazine, daunorubicin, decitabine, doxorubicin, epirubicin, etoposide, idarubicin, ifosfamide, irinotecan, lomustine, mechlorethamine, melphalan, mitomycin C, mitoxantrone, oxaliplatin, temozolomide, and topotecan.

The major limiting factor for determining the dose of radiation for the treatment of cancer is damage to normal tissue. As a result the treatment regimens are often below the curative dose. This is particularly the case for lungs, which are highly radiosensitive (Abratt, et al. (2002) Lung Cancer 36:225-233; Abratt & Morgan. (2002) Lung Cancer 35:103-109; Movsas, et al. (1997) Chest 111:1061-1076). The extent of radiation-induced fibrosis, and other toxicities, is dependent upon radiation dose, dose rate, fractionation schedule, and the total irradiated volume (Movsas, et al. (1997) Chest 111:1061-1076). Lung complications associated with radiation therapy have become more prevalent due to more aggressive radiation therapy and increased use of radiation therapy in combination with chemotherapy (Abid, et al. (2001) Curr. Opin. Oncol. 13:242-248). Studies have shown that patients receiving concurrent chemotherapy and radiation therapy for treatment of limited small cell lung carcinoma have improved survival. Follow up computed tomography studies after 1 year show that those who were irradiated with a dose greater than 30-35 Gy had an increased probability of lung fibrosis. If patients who received accelerated fractionation of twice daily are compared to those receiving a conventional single dose per day, there was a two-fold increase in probability of tissue damage (Rosen, et al. (2001) Radiology 221:614-622; Turrisi, et al. (1999) New Engl. J. Med. 340:265-271; Geara, et al. (1998) Int. J. Radiat. Oncol. Biol. Phys. 41:279-286). Therefore, there is a need for protection against radiation damage that would enable radiation therapy treatment of cancer within a curative radiation dose.

Accordingly, in some cases, a subject or patient in need of treatment is a subject suffering from, or at risk for, DNA damage by being exposed to or likely being exposed to ionizing radiation, e.g., at an acute dose of at least or about 0.1 Gy, e.g., at least or about 0.2 Gy, at least or about 0.5 Gy, at least or about 1 Gy, at least or about 2 Gy, at least or about 4 Gy, at least or about 5 Gy, at least or about 6 Gy, at least or about 7 Gy, at least or about 8 Gy, at least or about 10 Gy, at least or about 20 Gy, at least or about 30 Gy, at least or about 40 Gy, at least or about 50 Gy, or at least or about 100 Gy. Skilled practitioners will appreciate what levels of radiation damage DNA. In some cases, a subject will be, is being, or has been exposed to DNA-damaging levels of radiation, e.g., at least or about 0.1 Gy, e.g., at least or about 0.2 Gy, at least or about 0.5 Gy, at least or about 1 Gy, at least or about 2 Gy, at least or about 4 Gy, at least or about 5 Gy, at least or about 6 Gy, at least or about 7 Gy, at least or about 8 Gy, at least or about 10 Gy, at least or about 20 Gy, at least or about 30 Gy, at least or about 40 Gy, at least or about 50 Gy, or at least or about 100 Gy. The subject can be exposed to DNA-damaging levels of radiation through their occupation, e.g., a health care worker, miner, nuclear energy worker, and airline crew member, from a nuclear reactor, or from nuclear weapon, e.g., during warfare and/or an act of terrorism. The rPAI-1₂₃ can be administered before, while, and/or after the subject has been exposed to DNA-damaging levels of radiation.

In some cases, a subject in need of treatment is a patient suffering from or at risk for DNA damage by undergoing one or more genotoxic treatments, e.g., chemotherapy with a genotoxic agent, radiotherapy, or hyperthermia therapy. A patient suffering from or at risk for DNA damage may have a deleterious genetic defect or mutation. In some cases, the present methods ameliorate age-related damage to DNA in a patient.

In some cases, aging is a consequence of unrepaired DNA damage accumulation. Age-related damage to DNA results in DNA alteration that has an abnormal structure. Although both mitochondrial and nuclear DNA damage can contribute to aging, nuclear DNA is the main subject of this analysis. Nuclear DNA damage can contribute to aging either indirectly (by increasing apoptosis or cellular senescence) or directly (by increasing cell dysfunction).

In humans, DNA damage occurs frequently, and DNA repair processes have evolved to compensate. On average, approximately 800 DNA lesions occur per hour in each cell, or about 19,200 per cell per day. In any cell, some DNA damage may remain despite the action of repair processes. Accumulation of unrepaired DNA damage is more prevalent in certain types of cells, particularly in non-replicating or slowly replicating cells, which cannot rely on DNA repair mechanisms associated with DNA replication such as those in the brain, skeletal, and cardiac muscle. Older patients, e.g., at least or about 30 years old, at least or about 35 years old, at least or about 40 years old, at least or about 45 years old, at least or about 50 years old, at least or about 55 years old, at least or about 60 years old, at least or about 65 years old, at least or about 70 years old, at least or about 75 years old, or at least or about 80 years old, typically accumulate more age-related damage to DNA and can benefit from treatment to ameliorate age-related damage to DNA.

Skilled practitioners will appreciate that a patient can be diagnosed by a physician as suffering from or at risk for DNA damage and hence cellular senescence by any method known in the art. Individuals considered at risk for developing cellular senescence may benefit particularly from the invention, primarily because prophylactic treatment can begin before there is any evidence of cellular senescence. Individuals “at risk” include, e.g., subjects exposed to environmental, occupational, or therapeutic genotoxic agents. The skilled practitioner will appreciate that a patient can be determined to be at risk for DNA damage and cellular senescence by a physician's diagnosis.

As used herein, rPAI-1₂₃ is a recombinant plasminogen activator inhibitor-1 isoform that lacks the reactive center loop domain, located at amino acids 320-351, and lacks at least a portion of the heparin-binding domain. The sequence of human and porcine rPAI-1₂₃ are provided as SEQ ID NO:1 and SEQ ID NO:2, respectively.

| Human | |
|---|---|
| (SEQ ID NO: 1) | |
| MGPWNKDEIS TTDAIFVQRD LKLVQGFMPH FFRLFRSTVK QVDFSEVERA RFIINDWVKT | 60 |
| HTKGMISNLL GKGAVDQLTR LVLVNALYFN GQWKTPFPDS STHRRLFHKS DGSTVSVPMM | 120 |
| AQTNKFNYTE FTTPDGHYYD ILELPYHGDT LSMFIAAPYE KEVPLSALTN ILSAQLISHW | 180 |
| K | |
| Porcine | |
| (SEQ ID NO: 2) | |
| MGPWNKDEIS TADAIFVQRD LKLVQGFMPY FFRLFRTTVK QVDFSEMDRA RFIINDWVKR | 60 |
| HTKGMINDLL GQGAVDQLTR LVLVNALYFN GQWKTPFPEK STHHRLFHKS DGSTVSVPMM | 120 |
| AQTNKFNYTE FSTPDGHYYD ILELPYHGNT LSMFIAAPYE KEVPLSALTS ILDAQLISQW | 180 |
| K | |

The level of PAI-1 inhibition achieved by an rPAI-1₂₃ protein of this invention is in the range of from 10% to 100%, with a preferred level of PAI-1 inhibition of at least 25%.

In some embodiments, a full length rPAI-1₂₃ protein is used in the compositions and methods of the invention. In other embodiments, a truncated version of the rPAI-1₂₃ protein is used. The rPAI-1₂₃ protein can be truncated by removal of 1, 2, 3, 4, 5, 6, 7, 8, 9, 10, 11, 12, 13, 14, 15, 20, 30 or 35 amino acid residues from the N-terminus of a full length rPAI-1₂₃ protein disclosed herein and/or removal of 1, 2, 3, 4, 5, 6, 7, 8, 9, 10, 11, 12, 13, 14, 15, 20, 30 or 35 amino acid residues from the C-terminus of a full length rPAI-1₂₃ protein disclosed herein.

Amounts of rPAI-1₂₃ effective to reduce cellular senescence mediated by DNA damaging agents can be administered to a patient on the day the patient is exposed to a DNA damaging agent or any condition associated with DNA damage, or as having any risk factor associated with an increased likelihood that the patient will develop DNA damage (e.g., that the patient has recently been, is being, or will be exposed to a genotoxic agent). rPAI-1₂₃ can be administered for about 1, 2, 4, 6, 8, 10, 12, 14, 18, or 20 days, or greater than 20 days, e.g., 1 2, 3, 5, or 6 months, or until the patient no longer exhibits symptoms of cellular senescence, or until the patient is diagnosed as no longer being at risk for DNA damage. In a given day, rPAI-1₂₃ can be administered one or more times per day, continuously for the entire day, e.g., for up to 23 hours per day, e.g., up to 20, 15, 12, 10, 6, 3, or 2 hours per day, or up to 1 hour per day. An amount of rPAI-1₂₃ administered can be a mg/kg dose based on time and concentration of administration and a patient's body weight. Doses of from 0.1 to 2 μg/kg/day are envisioned as being a dose range for treatment of animals, including humans (e.g., having an average weight of 60 kg).

If the patient needs to be treated with a genotoxic drug (e.g., because prescribed by a physician), the patient can be treated with rPAI-1₂₃ before, during, and/or after administration of the drug. For example, rPAI-1₂₃ can be administered to the patient, intermittently or continuously, starting 0 to 20 days before the drug is administered (and where multiple doses are given, before each individual dose), e.g., starting at least about 30 minutes, e.g., about 1, 2, 3, 5, 7, or 10 hours, or about 1, 2, 4, 6, 8, 10, 12, 14, 18, or 20 days, or greater than days, before the administration. Alternatively or in addition, rPAI-1₂₃ can be administered to the patient concurrent with administration of the drug. Alternatively or in addition, rPAI-1₂₃ can be administered to the patient after administration of the drug, e.g., starting immediately after administration, and continuing intermittently or continuously for about 1, 2, 3, 5, 7, or 10 hours, or about 1, 2, 5, 8, 10, 20, 30, 50, or 60 days, indefinitely, or until a physician determines that administration of rPAI-1₂₃ is no longer necessary (e.g., after the genotoxic agent is eliminated from the body or can no longer cause DNA damage).

Accordingly, the present disclosure also features a method of administering a genotoxic treatment, e.g., administration of a genotoxic chemotherapeutic agent, radiotherapy, and hyperthermia therapy, to a patient. The method includes administering the genotoxic treatment to the patient, and before, during, and/or after administering the genotoxic treatment, administering to the patient a pharmaceutical composition comprising rPAI-1₂₃ in an amount effective to protect cells of the patient and/or reduce cellular senescence in the patient.

Beneficial effects of rPAI-1₂₃ can be assessed by morphological, phenotypic or genetic assays. For example, Senescent cells display a large flattened morphology and accumulate a senescence-associated β-galactosidase (SA-β-gal) activity that distinguishes them from most quiescent cells (Campisi & d'Adda di Fagagna (2007) Nat. Rev. Mol. Cell. Biol. 8:729-740)). β-galactosidase, a lysosomal hydrolase, is normally active at pH 4, but often in senescent cells β-galactosidase is active at pH 6. Thus, for example, one method to determine whether rPAI-1₂₃ protects against cellular senescence is to assay whether affected or diseased tissue stains positively for SA-β-Gal. For example, SA-β-Gal positive cells can be found in damaged or diseased or aging tissue, such as in skin, atherosclerotic plaque, pancreas, prostate, lung fibrosis, and liver fibrosis and cirrhosis. Treatment with rPAI-1₂₃ diminishes or reduces the number and/or intensity of SA-β-Gal positive cells.

Senescent cells also display abnormal genetic features. Normal human cells are diploid. Yet with each subcultivation, the percentage of polyploid cells, i.e., with three or more copies of chromosomes, increases. Mutations to the mitochondrial DNA (mtDNA) also appear to increase with age in vivo, though at low levels. For example, the first identified mutation was a deletion of 4,977 base pairs (bp) in the 16,569 by mtDNA. This deletion is observed both in vivo and in vitro. Thus, in some embodiments, senescent cells can be identified by screening for such genetic abnormalities and mutations. Thus, for example, another method to determine whether rPAI-1₂₃ protects against cellular senescence is to assay whether the affected or diseased tissue contains greater numbers of cells that are polyploid or have mutations in their mtDNA. Treatment with rPAI-1₂₃ diminishes or reduces the number of polyploid cells, number of cells having mutations in their mtDNA, and/or number of mtDNA mutations.

In addition, senescent cells often down-regulate genes involved in proliferation and extracellular matrix production, and upregulate inflammatory cytokines and other molecules known to modulate the microenvironment or immune response. Consistent with the role of cellular senescence as a barrier to malignant transformation, senescent cells activate the p53 and p16/Rb tumor suppressor pathways. p53 promotes senescence by transactivating genes that inhibit proliferation, including the p21/Cipl/Wafl cyclin-dependent kinase inhibitor and miR-34 class of microRNAs. In contrast, p16^{INK4a} promotes senescence by inhibiting cyclin-dependent kinases 2 and 4, thereby preventing Rb phosphorylation and allowing Rb to promote a repressive heterochromatin environment that silences certain proliferation-associated genes. Although the p53 and p16/Rb pathways act in parallel to promote senescence, their relative contribution to the program can be cell type-dependent. Thus, another method to determine whether rPAI-1₂₃ protects against cellular senescence is to assay whether cells in affected or diseased tissue activate the p53 and/or p16/Rb tumor suppressor pathways.

In certain embodiments of the invention, rPAI-1₂₃ reduces cellular senescence of lung tissue cells. In another embodiment, rPAI-1₂₃ reduces cellular senescence associated with radiation treatment of lung tissue. In accordance with this embodiment, lung tissue is contacted with an effective amount of rPAI-1₂₃ prior to, during or after radiation treatment.

In addition, cellular senescence and fibrosis is also associated with pulmonary hypertension, cardiac fibrosis following myocardial infarction, and systemic sclerosis (scleroderma). Therefore, in alternative embodiments, this invention provides a method for reducing cellular senescence in a subject having or at risk of having pulmonary hypertension, cardiac fibrosis following myocardial infarction, or systemic sclerosis (scleroderma).

For therapeutic use, rPAI-1₂₃ can be formulated with a pharmaceutically acceptable carrier, vehicle or excipient at an appropriate dose. Such pharmaceutical compositions can be prepared by methods and contain carriers which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000. A pharmaceutically acceptable carrier, composition or vehicle, such as a liquid or solid filler, diluent, excipient, or solvent encapsulating material, is involved in carrying or transporting the subject agent from one organ, or portion of the body, to another organ, or portion of the body. Each carrier must be acceptable in the sense of being compatible with the other ingredients of the formulation and not injurious to the patient.

Examples of materials which can serve as pharmaceutically acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Compositions of the present invention can be administered parenterally (for example, by intravenous, intraperitoneal, subcutaneous or intramuscular injection), topically, orally, intranasally, intravaginally, or rectally, according to standard medical practices.

The selected dosage level of rPAI-1₂₃ will depend upon a variety of factors including the route of administration, the time of administration, the duration of the treatment, other drugs, compounds and/or materials used in combination, the age, gender, weight, condition, general health and prior medical history of the patient being treated, and other factors well-known in the medical arts.

It is also contemplated that rPAI-1₂₃ can be used in the methods of the present invention either alone or in combination with other agents. Such agents can include, but are not be limited to, other PAI-1 inhibitors. One of skill in the art would choose which agents to use in combination based on their clinical experience with such agents, using doses approved for use in humans per the labeling for the drug products as marketed.

A physician having ordinary skill in the art can readily determine and prescribe the effective amount of the pharmaceutical composition required based upon the administration of similar compounds or experimental determination. For example, the physician could start doses of an agent at levels lower than that required in order to achieve the desired therapeutic effect and gradually increase the dosage until the desired effect is achieved. This is considered to be within the skill of the artisan and one can review the existing literature on a specific agent or similar agents to determine optimal dosing.

The following non-limiting examples are provided to further illustrate the present invention.

### Example 1

### rPAI-1₂₃ Increases Survival of Irradiated C57BL/6 Mice

Methods.

C57Bl/6 mice received intraperitoneal injections of rPAI-1₂₃ (5.4 μg/kg/day) or vehicle (PBS) for 18 weeks beginning two days prior to radiation exposure. Cohorts of mice treated with rPAI-1₂₃ or vehicle were exposed to thoracic irradiation in 5 daily fractions of 6 Gy (RT), and followed for survival (n=8 per group).

Results.

C57Bl/6 mice had increased survival in response to rPAI-1₂₃ treatment compared to control at 19 weeks post-radiation treatment (Table 1). Death due to dermatitis was not improved by rPAI-1₂₃ administration in the first 124 days post radiation.

TABLE 1

Percent Survival

| Days after RT | Vehicle + 6 Gy × 5 | rPAI-1₂₃ + 6 Gy × 5 |
|---|---|---|
| 0 | 100.0 | 100.0 |
| 20 | 100.0 | 100.0 |
| 40 | 87.5 | 100.0 |
| 60 | 87.5 | 100.0 |
| 63 | 87.5 | 87.5 |
| 69 | 87.5 | 87.5 |
| 94 | 75.0 | 62.5 |
| 108 | 75.0 | 62.5 |
| 115 | 62.5 | 62.5 |
| 124 | 62.5 | 62.5 |
| 130 | 37.5 | 62.5 |
| 145 | 37.5 | 62.5 |

### Example 2

### rPAI-1₂₃ Decreases Radiation-Induced Pulmonary Fibrosis in C57BL/6 Mice

Method.

Radiation exposure and rPAI-1₂₃ treatment were as described in Example 1. To assess fibrotic areas, sections of lung at 19 weeks post-irradiation were deparaffinized in xylene, and rehydrated through a graded alcohol series to water. Sections were then incubated in Bouin's picric-formalin and stained using Masson's trichrome with aniline blue as the collagen stain and Weigert's iron hematoxylin as the nuclear counterstain. In addition, the right lung (n=3 mice per group and condition) was weighed at the time of collection, mechanically homogenized, and snap frozen. Pulmonary hydroxyproline content was measured per sample after hydrolysis of a known weight of lung tissue (100 mg) in 1 ml of 6 N HCl at 110° C. for 18 hours. Hydrosylate was analyzed with the Biovision Hydroxyproline assay kit (Milpitas, Calif.) per manufacturer's instructions. The percent increase in pulmonary hydroxyproline per mouse was calculated based upon total lung weight and expressed as micrograms in the lung.

Results.

C57Bl/6 mice exposed to radiation therapy (RT)+vehicle had dense foci of subplueral fibrosis at 19 weeks, whereas the lungs of mice exposed to RT+rPAI-1₂₃ were largely devoid of fibrotic foci. Further, at 19 weeks after irradiation, hydroxyproline content was markedly decreased in mice that received rPAI-1₂₃ compared to vehicle (Table 2, p=0.001 between RT+vehicle and RT+rPAI-1₂₃).

TABLE 2

Hydroxyproline Content (μg/lung)

| Treatment | O Gy | 6 Gy × 5 |
|---|---|---|
| Vehicle | 39.8 ± 4.15 | 84.9 ± 11.79 |
| rPAI-1₂₃ | 45.6 ± 2.98 | 56.2 ± 10.79 |

### Example 3

### rPAI-1₂₃ Reduces Cellular Senescence in Response to Radiation

Method.

Radiation exposure and rPAI-1₂₃ treatment were as described in Example 1. Senescence-associated β-galactosidase (β-gal) activity was assessed with a commercially available assay kit (Abcam, Cambridge, Mass.) in lung tissues (n=3/group); and primary pneumocyte cultures according to established methods (Citrin, et al. (2013) J. Natl. Cancer Instit. 105:1474-1484). The percent of pro-surfactant C protein (SPC)-positive cells that also stained for β-gal was calculated based on visualization of both probes at 40× magnification in five separate fields per sample.

Results.

Cellular senescence in response to radiation was significantly decreased by rPAI-1₂₃ treatment in lung tissues with >2-fold reduction at 4, 8, and 16 weeks after radiation treatment (Table 3, p<0.001 at each time point), and in primary type 2 pneumocyte cultures with a 2-fold reduction at 5 days after radiation therapy (Table 4, p=0.036).

TABLE 3

| | | % X-Gal + SPC⁺ Cells | | |
|---|---|---|---|---|
| Treatment | | 4 Weeks | 8 Weeks | 16 Weeks |
| O Gy | Vehicle | 1.2 ± 1.65 | 1.6 ± 1.78 | 2.2 ± 3.95 |
| | rPAI-1₂₃ | 2.1 ± 3.51 | 5.1 ± 4.21 | 3.1 ± 3.62 |
| 6 Gy × 5 | Vehicle | 13.1 ± 1.34 | 17.6 ± 3.39 | 22.1 ± 6.93 |
| | rPAI-1₂₃ | 1.8 ± 1.78 | 6.5 ± 2.39 | 5.4 ± 4.17 |

TABLE 4

| | | % X-Gal + SPC⁺ Cells |
|---|---|---|
| Treatment | | (% in high power field) |
| O Gy | Vehicle | 12.2 ± 4.97 |
| | rPAI-1₂₃ | 5.0 ± 10.0 |
| 17.5 Gy | Vehicle | 34.7 ± 11.44 |
| | rPAI-1₂₃ | 15.7 ± 6.32 |

### Example 4

### rPAI-1₂₃ Treatment Reduces Collagen Deposition, but not Cell Proliferation in Murine Fibroblasts

Method.

Murine fibroblast NIH-3T3 cells (ATCC, Manassas, Va.) were grown in DMEM containing 10% FCS and 0.1% β-mercaptoethanol. Hydroxyproline content in fibroblast cultures was assayed in adherent cells scraped into ice cold PBS and pelleted by centrifugation. Cell pellets were hydrolyzed with 6 N HCl at 110° C. for 18 hours. Hydroxyproline was measured in hydrosylate using a Biovision Hydroxyproline assay kit (Milpitas, Calif.) per manufacturer's instructions. Fibroblast proliferation in response to rPAI-1₂₃ vs. vehicle was determined by antibody detection of BrdU concentration per test sample using a BrdU proliferation kit (EMD Millipore, Billerica, Mass.), following the manufacturer's instruction. Results are presented as BrdU concentration as determined at OD₄₅₀.

Results.

Treatment of NIH-3T3 fibroblasts with rPAI-1₂₃ resulted in an 18.5% (0.6 nM) and 25% (10 nM) decrease in collagen production (Table 5). However, rPAI-1₂₃ had no effect on proliferation compared to the control (Table 6).

TABLE 5

| | Hydroxyproline |
|---|---|
| rPAI-1₂₃ (nM) | (μg/ml/10⁴ cells) |
| 0 | 14.1 ± 0.70 |
| 0.6 | 11.5 ± 0.48 |
| 10 | 10.6 ± 0.84 |

TABLE 6

| rPAI-1₂₃ (nM) | Relative Proliferation |
|---|---|
| 0 | 1.0 ± 0.05 |
| 0.6 | 0.97 ± 0.09 |
| 10 | 0.90 ± 0.10 |

What is claimed is:

1. A method for reducing cellular senescence mediated by a DNA damaging agent comprising administering to a subject in need thereof an effective amount of rPAI-1₂₃ thereby reducing cellular senescence.

2. The method of claim 1, wherein the subject has been exposed to, is at risk of being exposed to, or will be exposed to a DNA damaging agent.

3. The method of claim 2, wherein the DNA damaging agent comprises radiation, a genotoxic agent, a mutagenic agent, or an aging or aging-related disorder.

4. The method of claim 1, wherein the rPAI-1₂₃ comprises SEQ ID NO:1 or SEQ ID NO:2.


SEQUENCE LISTING (2 sequences)

SEQ ID NO: 1 - protein, 181 aa, Artificial sequence
  Synthetic polypeptide
MGPWNKDEISTTDAIFVQRDLKLVQGFMPHFFRLFRSTVKQVDFSEVERARFIINDWVKTHTKGMISNLLGKGAVDQLTRLVLVNALYFNGQWKTPFPDSSTHRRLFHKSDGSTVSVPMMAQTNKFNYTEFTTPDGHYYDILELPYHGDTLSMFIAAPYEKEVPLSALTNILSAQLISHWK

SEQ ID NO: 2 - protein, 181 aa, Artificial sequence
  Synthetic polypeptide
MGPWNKDEISTADAIFVQRDLKLVQGFMPYFFRLFRTTVKQVDFSEMDRARFIINDWVKRHTKGMINDLLGQGAVDQLTRLVLVNALYFNGQWKTPFPEKSTHHRLFHKSDGSTVSVPMMAQTNKFNYTEFSTPDGHYYDILELPYHGNTLSMFIAAPYEKEVPLSALTSILDAQLISQWK